(19) United States
(12) Patent Application Publication
(10) Pub. No.: US 2021/0038093 A1
(43) Pub. Date: Feb. 11, 2021

(54) SYSTEM AND METHOD FOR BLOOD FLOW ASSESSMENT IN ARTERIOVENOUS FISTULA

(71) Applicant: CardiacM CO., Ltd., Zhunan Township (TW)

(72) Inventors: Teh-Ho TAO, Zhunan Township (TW); Tai-Been CHEN, Kaohsiung City (TW)

(73) Assignee: CardiacM CO., Ltd., Zhunan Township (TW)

(21) Appl. No.: 16/985,951

(22) Filed: Aug. 5, 2020

(30) Foreign Application Priority Data
Aug. 7, 2019 (TW) 108128122

(51) Int. Cl. A61B 5/0265; G06K 9/00; G06K 9/62; A61B 5/05; A61B 5/00

(52) U.S. Cl. CPC A61B 5/0265; G06K 9/00536; G06K 9/00503; G06K 9/6217; G01F 1/663; A61B 5/004; A61B 5/7225; A61B 5/7264; A61B 5/7278; A61B 5/05

(57) ABSTRACT

A method for blood flow assessment in an arteriovenous (AV) fistula includes steps of: emitting a carrier radio wave toward the AV fistula, and receiving a return wave signal; generating a transmission signal based on the return wave signal; recovering a digital signal from the transmission signal, performing a digital filtering process on the digital signal to result in a filtered signal, and generating a plurality of graphic files based on a waveform of the filtered signal; and performing image recognition on the graphic files, and outputting a result of the image recognition as a result of the blood flow assessment of the AV fistula.

### CROSS-REFERENCE TO RELATED APPLICATION

This application claims priority of Taiwanese Invention Patent Application No. 108128122, filed on Aug. 7, 2019.

### FIELD

The disclosure relates to a system and a method for blood flow assessment, and more particularly to a system and a method for blood flow assessment in an arteriovenous (AV) fistula.

### BACKGROUND

An arteriovenous (AV) fistula, which is a passageway between an artery and a vein, is usually surgically created for hemodialysis treatments and in a patient with chronic renal disease (CRD). In order to prevent adverse effect caused by occurrence of AV fistula occlusion, blood flow assessment in AV fistula is necessary.

Conventionally, HD03 hemodialysis monitors produced by Transonic Systems Inc. are used for blood flow assessment in AV fistula. However, such conventional assessment is intrusive and requires inserting two needles into the blood vessel(s) of a patient. Moreover, although it is able to provide high degree of accuracy, such conventional assessment is costly because of high-priced equipment (i.e., the HD03 hemodialysis monitors) and consumables (e.g., needles or tubing). For this reason, it is unsuitable for regular check-ups.

### SUMMARY

Therefore, an object of the disclosure is to provide a system and a method for blood flow assessment in an arteriovenous (AV) fistula that can alleviate at least one of the drawbacks of the prior art.

According to one aspect of the disclosure, the system includes a radio device and a processing device.

The radio device includes a transmitting antenna, a receiving antenna, a transmitting module and a receiving module.

The transmitting module is configured to cooperate with the transmitting antenna to emit a carrier radio wave to the AV fistula.

The receiving module is configured to receive, via the receiving antenna, a return wave signal which is formed through reflection of the carrier radio wave by the AV fistula, and to output a transmission signal which is generated based on the return wave signal.

The processing device includes a communication module, a digital filtering module and a recognition module.

The communication module is in signal connection with the receiving module, and is configured to receive the transmission signal, and to recover a digital signal from the transmission signal.

The digital filtering module is in signal connection with the differentiation module, and is configured to receive the digital signal, to perform a digital filtering process on the digital to result in a filtered signal, and to output the filtered signal.

The recognition module is in signal connection with the digital filtering module, and is configured to receive the filtered signal, to generate a plurality of graphic files based on a waveform of the filtered signal, to perform image recognition on the graphic files, and to output a result of the image recognition as a result of the blood flow assessment of the AV fistula.

According to another aspect of the disclosure, the method is adapted to be implemented by the system that is previously described. The method includes steps of:
A) emitting a carrier radio wave toward the AV fistula, and receiving a return wave signal which is formed through reflection of the carrier radio wave by the AV fistula;
B) generating a transmission signal based on the return wave signal;
C) recovering a digital signal from the transmission signal, performing a digital filtering process on the digital signal to result in a filtered signal, and generating a plurality of graphic files based on a waveform of the filtered signal; and
D) performing image recognition on the graphic files, and outputting a result of the image recognition as a result of the blood flow assessment of the AV fistula.

### BRIEF DESCRIPTION OF THE DRAWINGS

Other features and advantages of the disclosure will become apparent in the following detailed description of the embodiment with reference to the accompanying drawings, of which:

FIG. 1 is a schematic diagram illustrating an embodiment of measurement performed by a system for blood flow assessment in an arteriovenous (AV) fistula according to the disclosure;

FIG. 2 is a block diagram illustrating a first embodiment of the system according to the disclosure;

FIG. 3 is a schematic diagram illustrating an exemplary waveform of a bipolar pulse oscillation wave generated by the system according to the disclosure;

FIG. 4 is a flow chart illustrating a first embodiment of a method for blood flow assessment in an AV fistula according to the disclosure;

FIG. 5 is a flow chart illustrating an embodiment of sub-steps of generating graphic files in the method according to the disclosure;

FIGS. 6 to 11 are schematic diagrams cooperatively illustrating an embodiment of obtaining the graphic files according to the disclosure;

FIG. 12 is a schematic diagram illustrating a pressure model of an AV fistula to assist in the explanation of the system and the method according to the disclosure;

FIG. 13 is a block diagram illustrating a second embodiment of the system according to the disclosure;

FIG. 14 is a flow chart illustrating a second embodiment of the method according to the disclosure; and

FIGS. 15 to 17 are schematic diagrams cooperatively illustrating an example of practicing the second embodiment of the method for blood flow assessment according to the disclosure.

### DETAILED DESCRIPTION

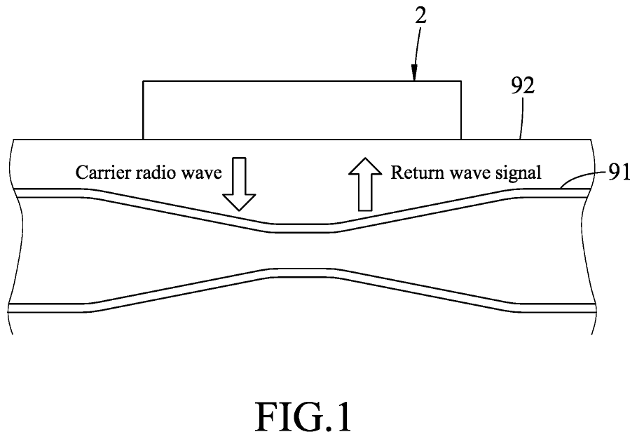

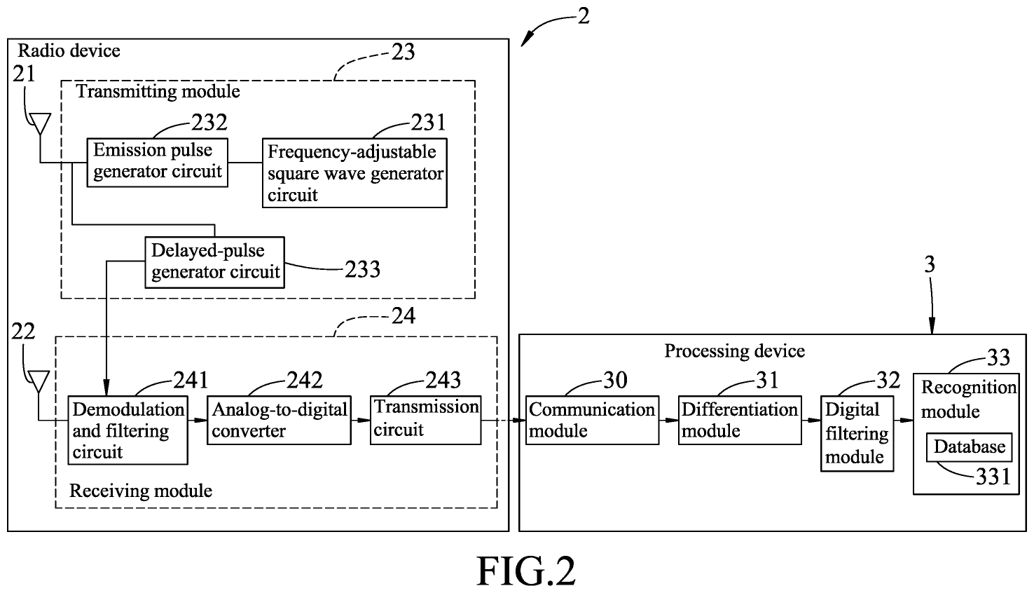

Referring to FIGS. 1 to 3, a first embodiment of a system for blood flow assessment in an arteriovenous (AV) fistula according to the disclosure is illustrated. The system includes a radio device 2 and a processing device 3.

As shown in FIG. 1, the radio device 2 is adapted to be disposed on the skin 92 above an AV fistula 91 of a subject. The radio device 2 includes a transmitting antenna 21, a receiving antenna 22, a transmitting module 23 and a receiving module 24.

The transmitting module 23 is configured to cooperate with the transmitting antenna 21 to emit a carrier radio wave toward the AV fistula 91. The receiving module 24 is configured to receive, via the receiving antenna 22, a return wave signal which is formed through reflection of the carrier radio wave by the AV fistula 91, and to output a transmission signal which is generated based on the return wave signal.

Specifically speaking, the transmitting module 23 includes a frequency-adjustable square wave generator circuit 231, an emission pulse generator circuit 232 and a delayed-pulse generator circuit 233. The emission pulse generator circuit 232 is electrically connected between the transmitting antenna 21 and the frequency-adjustable square wave generator circuit 231. The delayed-pulse generator circuit 233 is electrically connected to the transmitting antenna 21.

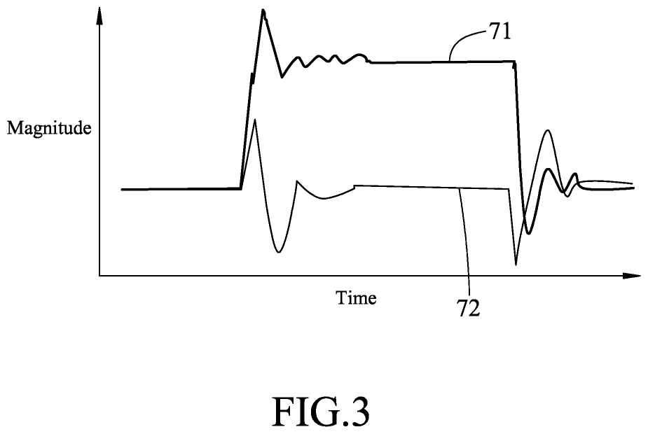

Referring to FIG. 3, the frequency-adjustable square wave generator circuit 231 is configured to generate a square wave, and output the square wave to the emission pulse generator circuit 232. The square wave thus generated is adjustable to have a frequency ranging from 125 KHz to 4 MHz.

The emission pulse generator circuit 232 is implemented by complementary metal-oxide-semiconductor (CMOS) transistors, but implementation of the emission pulse generator circuit 232 is not limited to the disclosure herein and may vary in other embodiments. High-pass filtering effect caused by gate-drain coupling capacitors of the CMOS transistors in the emission pulse generator circuit 232 results in transient damped oscillation at the rising edge and the falling edge of the square wave generated by the frequency-adjustable square wave generator circuit 231. The square wave is exemplified by a waveform 71 shown in FIG. 3. The time interval of the damped oscillation may range from 5 to 8 ns.

In this embodiment, the transmitting antenna 21 is implemented by a wideband patch antenna. It is worth to note that the square wave, which has damped oscillations, is fed into the transmitting antenna 21 and excites the TM₀₁ resonant mode, which is a kind of first order transverse mode of electromagnetic radiation, in the transmitting antenna 21. In addition, the transmitting antenna 21, having a 200 MHz frequency bandwidth, works as a band-pass filter to block the direct-current (DC) component and to pass the high frequency component (i.e., the damped oscillations). As a result, a bipolar pulse oscillation wave is emitted by the transmitting antenna 21 as the carrier radio wave shown in FIG. 3. At the same time, when the delayed-pulse generator circuit 233 receives the square wave with damped oscillation from the emission pulse generator circuit 232, the DC component of the square wave with damped oscillation is removed by a band-pass filter of the delayed-pulse generator circuit 233 to result in the bipolar pulse oscillation wave, and then the delayed-pulse generator circuit 233 is configured to delay the bipolar pulse oscillation wave to result in a delayed bipolar pulse oscillation wave, and to output the delayed bipolar pulse oscillation wave to the receiving module 24.

The carrier radio wave emitted by the transmitting antenna 21 would penetrate the skin 92 of the subject, and then be reflected by a surface of the AV fistula 91 of the subject. Because of the Doppler effect, periodic movement of the surface of the AV fistula 91 due to arterial pulsation would change the frequency of the carrier radio wave reflected by the surface of the AV fistula 91. That is to say, the frequency of the return wave signal thus formed would be different from that of the carrier radio wave and would contain information on the periodic movement and displacements of the AV fistula 91.

The receiving module 24 includes a demodulation and filtering circuit 241, an analog-to-digital converter 242 and a transmission circuit 243.

The demodulation and filtering circuit 241 is electrically connected to the receiving antenna 22 and the delayed-pulse generator circuit 233. The demodulation and filtering circuit 241 is configured to receive the return wave signal via the receiving antenna 22, and to receive the delayed bipolar pulse oscillation wave from the delayed-pulse generator circuit 233. The demodulation and filtering circuit 241 is further configured to perform demodulation on the return wave signal by using the delayed bipolar pulse oscillation wave, and to perform band-pass filtering to remove high frequency noise from the return wave signal which has undergone the demodulation so as to result in a demodulated signal. Thereafter, the demodulation and filtering circuit 241 is configured to output the demodulated signal. It should be noted that the demodulated signal contains information on the displacements of the surface of the AV fistula 91.

The analog-to-digital converter 242 is electrically connected to the demodulation and filtering circuit 241. The analog-to-digital converter 242 is configured to receive the demodulated signal, to perform an analog-to-digital conversion on the demodulated signal to result in a digital signal, and to output the digital signal.

The transmission circuit 243 is electrically connected to the analog-to-digital converter 242. The transmission circuit 243 is configured to receive the digital signal, to transform the digital signal into the transmission signal, and to output the transmission signal. In this embodiment, transmission of the transmission signal by the transmission circuit 243 is implemented by Bluetooth wireless techniques. It should be noted that the information on the displacements of the surface of the AV fistula 91 is kept in the transmission signal.

The processing device 3 includes a communication module 30, a differentiation module 31, a digital filtering module 32 and a recognition module 33. In this embodiment, the communication module 30, the differentiation module 31 and the digital filtering module 32 are implemented together by a smartphone, and more specifically, a transceiver, a microprocessor and/or a digital signal processor included in the smartphone; the recognition module 33 is implemented by a server. However, implementation of the processing device 3 is not limited to the disclosure herein and may vary in other embodiments. For example, the processing device 3 may be implemented entirely by a single server.

The communication module 30 is in signal connection with the transmission circuit 243 of the receiving module 24. The communication module 30 is configured to receive the transmission signal, and to recover the digital signal from the transmission signal. In this embodiment, receipt of the transmission signal by the communication module 30 is implemented by Bluetooth wireless techniques.

The differentiation module 31 is in signal connection with the communication module 30. The differentiation module 31 is configured to receive the digital signal, to perform differentiation on the digital signal with respect to time, and to output the digital signal thus differentiated.

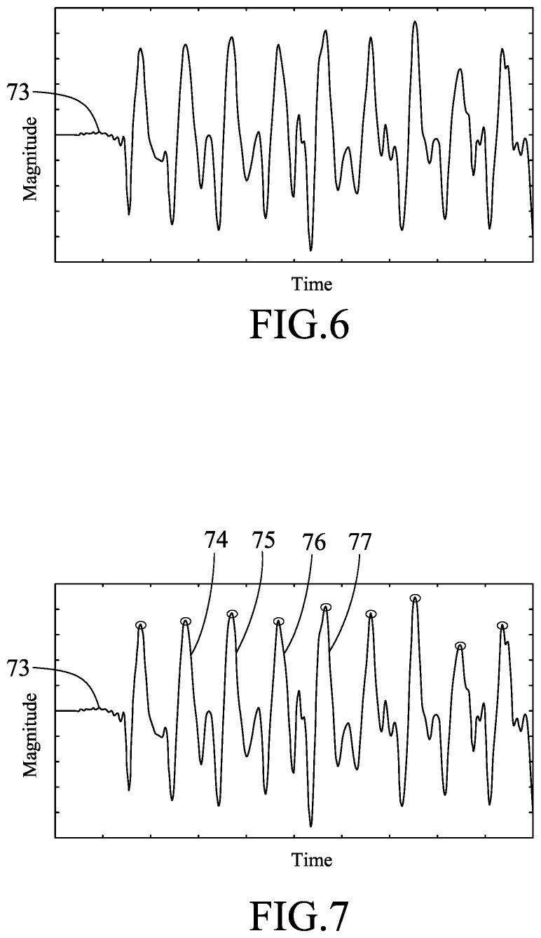

The digital filtering module 32 is in signal connection with the differentiation module 31. The digital filtering module 32 is configured to receive the digital signal thus differentiated, to perform a digital filtering process on the digital signal thus differentiated to result in a filtered signal exemplified by a waveform 73 shown in FIG. 6, and to output the filtered signal. In this embodiment, the digital filtering module 32 is a finite impulse response (FIR) filter that is realized by a mobile application (APP) installed on the smartphone. The digital filtering module 32 is configured to pass a part of the digital signal thus differentiated with a frequency ranging from 0.2 Hz to 10 Hz as the filtered signal.

It should be noted that the information on the displacements of the surface of the AV fistula 91 is kept in the filtered signal. In this embodiment, a copy of the information on the displacements of the surface of the AV fistula 91 is stored in the smartphone that embodies the communication module 30, the differentiation module 31 and the digital filtering module 32. Moreover, the information on the displacements of the surface of the AV fistula 91 can be displayed on a screen of the smartphone.

The recognition module 33 is in signal connection with the digital filtering module 32. In one embodiment, the recognition module 33 and the digital filtering module 32 are capable of communicating with each other by using wireless communication or network technologies, such as global system for mobile communications (GSM), other generations of wireless mobile telecommunications technology, Wi-Fi, Bluetooth or the like. The recognition module 33 is configured to receive the filtered signal from the digital filtering module 32, to generate a plurality of graphic files based on the waveform of the filtered signal, to perform image recognition on the graphic files, and to output a result of the image recognition as a result of the blood flow assessment of the AV fistula 91.

In this embodiment, the recognition module 33 includes a database 331 that stores in advance a convolutional neural network (CNN) model, and the recognition module 33 is configured to perform image recognition on the graphic files by using the CNN model. Specifically, in one embodiment, the recognition module 33 is configured to obtain a Visual Geometry Group-19 (VGG-19) model from the Internet, and to perform image recognition on the graphic files by using the VGG-19 model. However, image recognition may be implemented differently in other embodiments.

It should be noted that in order to use the CNN model, e.g., the VGG-19 model, in performing image recognition, the VGG-19 model has to be trained in advance by using a large number of graphic files that are associated with blood flows in AV fistulas, assessment results of which have been verified by an HD03 hemodialysis monitor produced by Transonic Systems Inc. Each of a training data set and a test data set includes two hundred graphic files, half of which corresponds to abnormal subjects (who have been diagnosed with AV fistula stenosis) and the other half of which corresponds to normal subjects where the abnormal and normal subjects have been verified by the HD03 hemodialysis monitor. At first, the VGG-19 model is trained by the training data set. Subsequently, the VGG-19 model is tested by the test data set on aspects of sensitivity (also called the true positive rate) and specificity (also called the true negative rate) of the performance of image recognition. When the sensitivity and the specificity of the VGG-19 model thus tested are both greater than 0.9, it is determined that the VGG-19 model has been trained up to enough predictability.

Referring to FIGS. 1, 4 and 5, a first embodiment of a method for blood flow assessment in an AV fistula according to the disclosure is illustrated. The method is adapted to be implemented by the system that is previously described. The method includes steps 81 to 84 delineated below.

In step 81, the system emits the carrier radio wave toward the AV fistula 91, and receives the return wave signal which is formed through reflection of the carrier radio wave by the AV fistula 91.

In step 82, the system generates the transmission signal based on the return wave signal.

In step 83, the system recovers the digital signal from the transmission signal, performs differentiation on the digital signal with respect to time, performs the digital filtering process on the digital signal thus differentiated to result in the filtered signal, and generates the graphic files based on the waveform of the filtered signal.

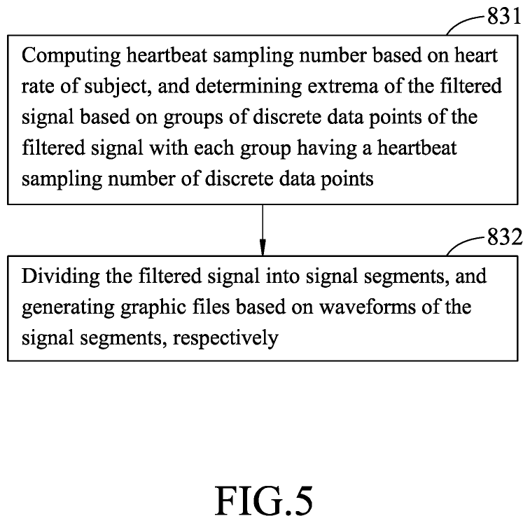

Referring to FIG. 5, with respect to generating the graphic files, step 83 further includes the following sub-steps 831 and 832.

In sub-step 831, the system computes a heartbeat sampling number based on a heart rate of the subject, where the heartbeat sampling number is a number of sample values in a time interval that corresponds to one heartbeat. Then, for every heartbeat sampling number of discrete data points of the filtered signal, where the discrete data points correspond to the sample values of the filtered signal, the system determines an extremum of the filtered signal from among the discrete data points. In this embodiment, for each specific segment (i.e., for every heartbeat sampling number of the discrete data points) of the filtered signal, the extremum is a relative maximum within the specific segment.

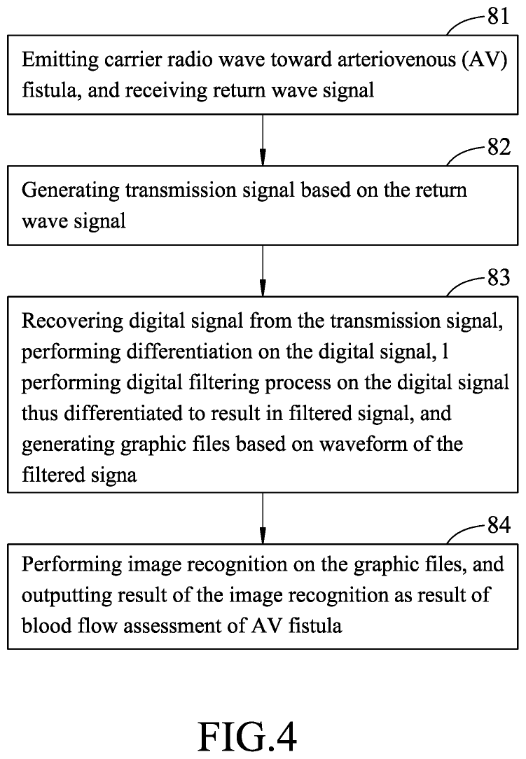

Specifically speaking, referring to FIGS. 4 to 11, assuming that the heart rate of the subject is 80 beats per minute (bpm) and that the sampling rate for the system to process the digital signal is 128 Hz (i.e., the filtered signal includes 128 sample values every second), the heartbeat sampling number computed by the system would be

$$\frac{128}{80 \text{/} 60} = 96 ,$$

meaning 96 sample values would be recorded for each heartbeat. Subsequently, the system determines, for each heartbeat, the extremum of the filtered signal by selecting the relative maximum of the 96 sample values of the filtered signal to serve as the extremum. As shown in FIGS. 6 and 7, parts of the filtered signal 73 that are marked with circles are the extrema of the filtered signal 73.

In one embodiment, the system further determines, for every heartbeat sampling number of the sample values, whether the extremum is valid or invalid based on a result of determination as to whether the value of the extremum is greater than an average of the magnitudes of the heartbeat sampling number of the sample values (i.e., the 96 sample values) by 1.95 times of a standard deviation of the magnitudes of the heartbeat sampling number of the sample values. The system determines that the extremum is valid when the result of the determination is affirmative, and determines that the extremum is invalid when the result of the determination is negative.

In sub-step 832, the system divides, based on the extrema thus determined, the filtered signal into a plurality of signal segments, and generates the graphic files based on waveforms of the signal segments, respectively.

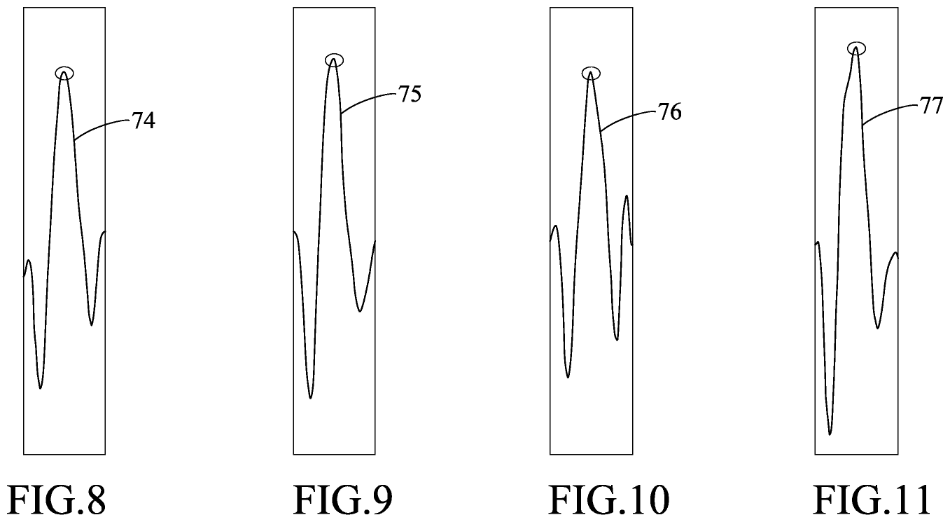

Specifically speaking, by cutting the filtered signal at cut-points, each of which corresponds to a midpoint on a time axis between corresponding two adjacent extrema, the system divides the filtered signal into the signal segments. Subsequently, the system converts the waveform of each of the signal segments into the respective one of the graphic files. For example, the waveforms 74 to 77 of the signal segments shown in FIGS. 8 to 11 are obtained from the filtered signal 73 shown in FIG. 7 by cutting the filtered signal 73 at five cut-points which are midpoints each between corresponding two adjacent extrema, starting from the first to sixth extrema (from left to right) shown in FIG. 7. The system then generates four graphic files based on the waveforms 74 to 77 of the signal segments as shown in FIGS. 8 to 11.

In one embodiment, the system deletes any graphic file that corresponds to a signal segment with an invalid extremum, so the graphic file thus deleted is not processed in step 84.

In step 84, the system performs image recognition on the graphic files, and outputs the result of the image recognition as the result of the blood flow assessment of the AV fistula 91. In one embodiment, the system performs image recognition on the graphic files by using the CNN model. In one embodiment, the system performs image recognition on the graphic files by using the VGG-19 model.

The result of the image recognition may be provided to a user in a form of a combination of a text message and a numerical value, such as “Probability of being abnormal: 0.89” or “Probability of being normal: 0.11”. When the probability of being abnormal is greater than a predetermined value (e.g., 0.7), an alert is given to notify the user that the result of the blood flow assessment is abnormal. In this way, medical professionals being alerted may determine whether to perform any further assessment.

The principle behind this disclosure will be described in the following paragraphs.

Referring to FIGS. 1, 2 and 12, the carrier radio wave emitted toward the AV fistula 91 is influenced by vibration (i.e., periodic movement) of the AV fistula 91, so the return wave signal, which is formed through reflection of the carrier radio wave by the AV fistula 91, contains information on the vibration of the AV fistula 91 under the Doppler effect.

On the assumption that

$$\frac{4 π {R}_{0}}{{λ}_{s} t} = ( 2 n + 1 ) \times \frac{π}{2}$$

and that 4πνₛt sin ωₛt<<λₛ, where R₀ represents the initial distance between the transmitting antenna 21 and the AV fistula 91, λₛ represents the wavelength of the carrier radio wave travelling through the skin and subcutaneous tissue, t represents time, n is an integer, π is the circular constant, νₛ represents the velocity of the movement of the AV fistula 91, and ωₛ represents the angular frequency of the movement of the AV fistula 91, the magnitude of the low frequency part of the return wave signal is directly proportional to the variation of the distance between the transmitting antenna 21 and the AV fistula 91. Such proportional relationship can be expressed as

$$\begin{matrix}B ( t ) ∝ \frac{4 π {ν}_{s} t sin {ω}_{s} t}{{λ}_{s}} = \frac{4 π ( {R}_{0} - R ( t ) )}{{λ}_{s}} = \frac{4 π Δ R}{{λ}_{s}} , or \\ B ( t ) ∝ Δ R ,\end{matrix}$$

where B(t) represents the magnitude of the low frequency part of the return wave signal, R(t)=R₀−νₛt sin ωₛt represents the distance between the transmitting antenna 21 and the AV fistula 91 with respect to time, and ΔR represents the variation of the distance between the transmitting antenna 21 and the AV fistula 91.

Since the variation of the distance (ΔR) between the transmitting antenna 21 and the AV fistula 91 can be regarded as the variation of the radius Δr of the AV fistula 91, the magnitude of the low frequency part of the return wave signal is also directly proportional to the variation of the radius of the AV fistula 91, i.e., B(t) ∝ Δr.

Additionally, the stiffness S of the AV fistula 91 can be expressed as

$$S = \frac{Δ P \cdot V}{Δ V} ,$$

where ΔP represents the blood pressure on the AV fistula 91, V=πr² represents the volume of the AV fistula 91 per unit length, ΔV=2πr·Δr represents the variation of the volume of the AV fistula 91 per unit length, and r represents the radius of the AV fistula 91. That is to say, the blood pressure on the AV fistula 91 is proportional to the variation of the radius of the AV fistula 91, i.e.,

$$Δ P = S \cdot \frac{2 Δ r}{r} ∝ Δ r .$$

Based on the aforementioned derivations, the magnitude of the low frequency part of the return wave signal is directly proportional to the blood pressure on the AV fistula 91, i.e., B(t) ∝ Δr ∝ ΔP.

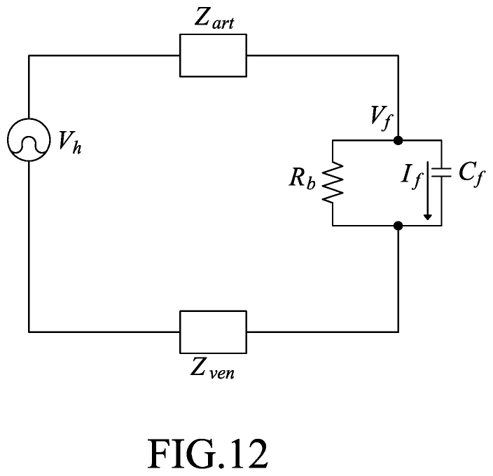

FIG. 12 illustrates the pressure model of an AV fistula, wherein νₕ represents the left ventricular pressure, zₐᵣₜ represents the impedance of the artery, zᵥₑₙ represents the impedance of the vein, R_{b} represents the fistula branch resistance, C_{f} represents the fistula compliance, V_{f} represents the fistula pressure (i.e., the blood pressure ΔP on the AV fistula 91) , and I_{f} represents the fistula flow (i.e., the blood flow in the AV fistula 91). Normally, the fistula branch resistance R_{b} can be regarded as infinity, and will be omitted in the following derivations.

The fistula flow I_{f} is a function of the fistula pressure V_{f}, and can be formulated as

$${I}_{f} = {C}_{f} \frac{d {V}_{f}}{dt} = {C}_{f} \frac{d Δ P}{dt} .$$

In particular, the fistula flow I_{f} is proportional to the derivative of the blood pressure on the AV fistula 91 with respect to time, i.e.,

$${I}_{f} ∝ \frac{d Δ P}{dt} .$$

Based on the proportional relationship between the magnitude of the low frequency part of the return wave signal and the blood pressure on the AV fistula 91, the fistula flow I_{f} is proportional to the derivative of the magnitude of the low frequency part of the return wave signal with respect to time, i.e.,

$${I}_{f} ∝ \frac{dB ( t )}{dt} .$$

By observing the magnitude of the low frequency part B(t) of the return wave signal, information on the blood pressure ΔP on the AV fistula 91 and the variation of the radius Δr of the AV fistula 91 can be obtained. However, information on the fistula flow I_{f} cannot be directly observed from the magnitude of the low frequency part B(t) of the return wave signal. The system and the method according to the disclosure calculate the derivative

$$\frac{dB ( t )}{dt}$$

of the magnitude of the low frequency part of the return wave signal with respect to time, and hence information on the fistula flow I_{f} can be obtained to realize the blood flow assessment of the AV fistula.

It is worth to note that AV fistula stenosis would cause blood flow velocity in a narrowing part of the AV fistula 91 to increase, resulting in turbulent flow downstream of the AV fistula 91. The turbulent flow would distort the waveform of the low frequency part B(t) of the return wave signal. Therefore, AV fistula stenosis may be detected by analyzing whether there is distortion in the waveform of the low frequency part B(t) of the return wave signal.

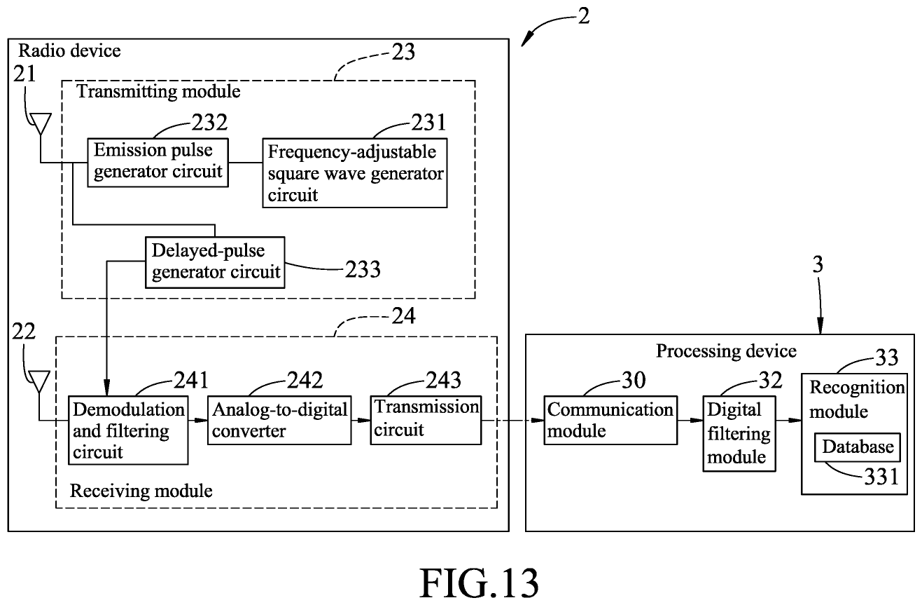

Referring to FIG. 13, a second embodiment of the system for blood flow assessment in an AV fistula according to the disclosure is illustrated. The second embodiment is similar to the first embodiment, but the differentiation module 31 is omitted in the second embodiment. In other words, the digital filtering module 32 is directly in signal connection with the communication module 30, and is configured to receive the digital signal that is not differentiated, to perform the digital filtering process on the digital signal to result in the filtered signal, and to output the filtered signal.

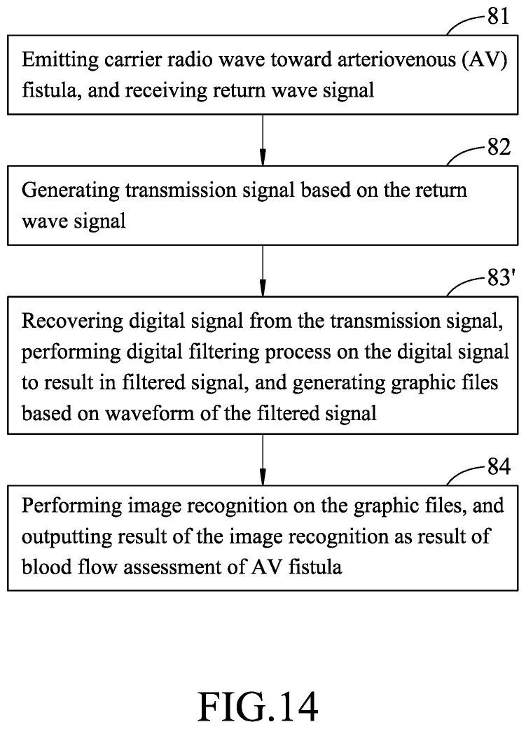

Referring to FIG. 14, a second embodiment of the method for blood flow assessment in an AV fistula according to the disclosure is illustrated. The second embodiment of the method is adapted to be implemented by the second embodiment of the system that is mentioned above, and is similar to the first embodiment of the method. However, performance of differentiation on the digital signal in step 83 of the first embodiment is omitted. That is to say, in step 83′ as shown in FIG. 14, the system recovers the digital signal from the transmission signal, performs the digital filtering process on the digital signal to result in the filtered signal, and generates the graphic files based on the waveform of the filtered signal. It is noted that the digital signal recovered from the transmission signal is not differentiated before being filtered.

Referring to Table 1 below, in an example of practicing the method for blood flow assessment according to the disclosure, results of blood flow assessment for forty-six subjects by using the second embodiment of the method are illustrated, and are compared with ground truth. The subjects respectively correspond to ID-14 to ID-59, and image recognition in step 84 (referred to as model prediction hereinafter) is performed on the graphic files generated with respect to the forty-six subjects. In the ground truth, a subject whose blood flow rate in AV fistula measured by the HD03 hemodialysis monitor is lower than 600 ml/min would be regarded as “abnormal” and would be diagnosed as having AV fistula stenosis.

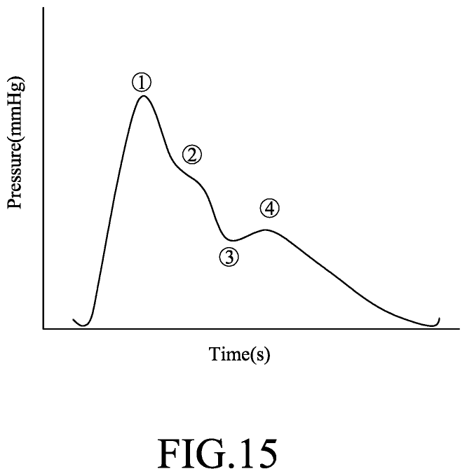

Referring to FIG. 15, a typical pulse wave obtained from a healthy adult is illustrated. Regarding the waveform of the typical pulse wave shown in FIG. 15, a portion marked with \textcircled{1} corresponds to the shock of left ventricular contraction, a portion marked with \textcircled{2} corresponds to the reflected wave, a portion marked with \textcircled{3} is the incisura by the sudden closing of aortic valve, and a portion marked with \textcircled{4} corresponds to the closure of aortic valve with consequent rebound of blood.

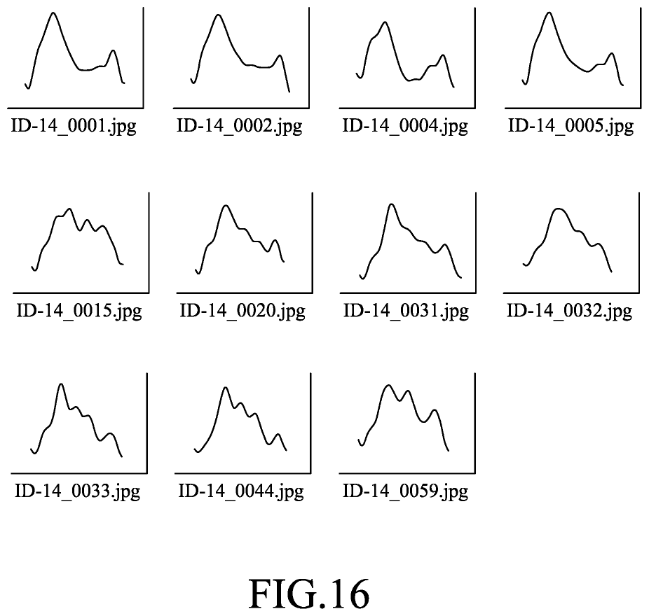

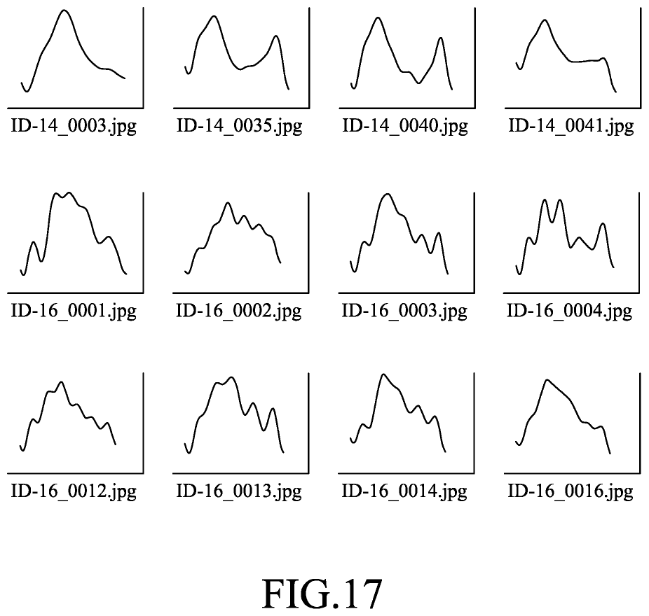

Generally, a majority of the waveforms in the graphic files generated based on the filtered signals for normal subjects should be similar to that of the typical pulse wave. Therefore, in the process of the model prediction, the waveform in the graphic file classified as “normal” should be more similar to the typical pulse wave than the waveform in the graphic file classified as “abnormal” is to the typical pulse wave. For example, FIG. 16 exemplarily shows waveforms that are obtained from the subjects corresponding to ID-14 and ID-16 and that are classified by the VGG-19 model as “normal”. Comparatively, FIG. 17 exemplarily shows waveforms that are also obtained from the subjects corresponding to ID-14 and ID-16 and that are classified by the VGG-19 model as “abnormal”.

TABLE 1

| | Abnormal | Normal | Noise | Flow |
|---|---|---|---|---|
| ID | Rate | Rate | Rate | (ml/min) |
| ID-14 | 0.07 | 0.93 | 0 | 910 |
| ID-15 | 0 | 1 | 0 | 880 |
| ID-16 | 0.85 | 0.12 | 0.03 | 350 |
| ID-17 | 0.69 | 0.13 | 0.18 | 990 |
| ID-18 | 0.31 | 0.4 | 0.28 | 820 |
| ID-19 | 0 | 1 | 0 | 1410 |
| ID-20 | 0.05 | 0.92 | 0.03 | 1380 |
| ID-21 | 0.52 | 0.48 | 0 | 530 |
| ID-22 | 0.78 | 0.01 | 0.21 | 590 |
| ID-23 | 0.14 | 0.86 | 0 | 610 |
| ID-24 | 0.4 | 0.48 | 0.12 | 630 |
| ID-25 | 0.08 | 0.92 | 0 | 1380 |
| ID-26 | 0.53 | 0.08 | 0.39 | 560 |
| ID-27 | 0.58 | 0.37 | 0.05 | 430 |
| ID-28 | 0.14 | 0.8 | 0.07 | 870 |
| ID-29 | 0.08 | 0.9 | 0.02 | 650 |
| ID-30 | 0.28 | 0.64 | 0.08 | 880 |
| ID-31 | 0.36 | 0.5 | 0.14 | 640 |
| ID-32 | 0.58 | 0.41 | 0.02 | 360 |
| ID-33 | 0.88 | 0.02 | 0.11 | 560 |
| ID-34 | 0.15 | 0.76 | 0.1 | 630 |
| ID-35 | 0.66 | 0.34 | 0 | 660 |
| ID-36 | 0.14 | 0.72 | 0.14 | 930 |
| ID-37 | 0.52 | 0.31 | 0.17 | 1970 |
| ID-38 | 0 | 1 | 0 | 3110 |
| ID-39 | 0.03 | 0.87 | 0.11 | 3440 |
| ID-40 | 0.5 | 0.39 | 0.11 | 670 |
| ID-41 | 0.36 | 0.05 | 0.14 | NA |
| ID-42 | 0.03 | 0.97 | 0 | 1120 |
| ID-43 | 0 | 0.94 | 0.06 | 2210 |
| ID-44 | 0.25 | 0.75 | 0 | 1210 |
| ID-45 | 0.31 | 0.65 | 0.04 | 1060 |
| ID-46 | 0.2 | 0.64 | 0.15 | 840 |
| ID-47 | 0.53 | 0.2 | 0.27 | 510 |
| ID-48 | 0.11 | 0.88 | 0.01 | 1250 |
| ID-49 | 0.06 | 0.39 | 0.56 | 710 |
| ID-50 | 0 | 1 | 0 | 1100 |
| ID-51 | 0.43 | 0.47 | 0.1 | 480 |
| ID-52 | 0.85 | 0.15 | 0 | 320 |
| ID-53 | 0.41 | 0.59 | 0 | 1430 |
| ID-54 | 0.72 | 0.28 | 0 | 1550 |
| ID-55 | 0.4 | 0.19 | 0.41 | 730 |
| ID-56 | 0.93 | 0 | 0.07 | 470 |
| ID-57 | 0 | 1 | 0 | 790 |
| ID-58 | 0.15 | 0.34 | 0.52 | 740 |
| ID-59 | 0 | 0.57 | 0.43 | 820 |

TP = 10; FN = 1; FP = 4; TN = 31

TABLE 2

| | Model Prediction (CNN Model: Vgg-19) | |
|---|---|---|
| Ground Truth | Abnormal | Normal |
| Abnormal | 10 | 1 |
| (Flow < 600) | | |
| Normal | 4 | 31 |
| (Flow ≥ 600) | | |

Sensitivity = TP/(TP + FN) = 0.91
Specificity = TN/(TN + FP) = 0.89

Referring to Table 2 above, among the forty-six subjects, ten abnormal subjects (i.e., IDs-16, 21, 22, 26, 27, 32, 33, 47, 52 and 56) are determined by the model prediction as “abnormal”, and such result is regarded as “true positive (TP)”; one abnormal subject (i.e., ID-51) is determined by the model prediction as “normal”, and such result is regarded as “false negative (FN)”; four normal subjects (i.e., IDs-17, 35, 37 and 54) are determined by the model prediction as “abnormal”, and such result is regarded as “false positive (FP)”; thirty-one normal subjects are determined by the model prediction as “normal”, and such result is regarded as “true negative (TN)”. Therefore, the sensitivity of the blood flow assessment made by the model prediction is 0.91, and the specificity of the assessment made by the model prediction is 0.89. In other words, the blood flow assessment made by the second embodiments of the method and the system according to the disclosure is accurate.

In summary, by performing image recognition on graphic files generated based on the filtered signal that contains information on reflection of the carrier radio wave by the AV fistula, the system and the method according to the disclosure can carry out the blood flow assessment of the AV fistula.

The approach adopted by the disclosure is non-intrusive, and thus is convenient to use. In addition, the cost incurred in such approach is much lower than purchasing the HD03 hemodialysis monitor produced by Transonic Systems Inc., and no consumable is required. Therefore, the method and the system for blood flow assessment in the AV fistula according to the disclosure are suitable for regular check-ups in hospitals and home heath care.

When the result of the blood flow assessment of the AV fistula generated by the method or the system according to the disclosure shows an abnormal result (e.g., AV fistula stenosis), the HD03 hemodialysis monitor produced by Transonic Systems Inc. or other instruments may then be adopted to perform more accurate assessment. In this way, stenosis in the AV fistula may be detected in the early phase before occurrence of vascular obstruction, and medical professionals may perform appropriate treatment (e.g., percutaneous transluminal angioplasty, PTA) in time. Hence, effect of hemodialysis may be improved, re-admission rate may be reduced, medical expenditure may be reduced, and quality of life may be enhanced.

Further, results of the blood flow assessment obtained by using the method and the system according to the disclosure may be compared with results of conventional intrusive assessment (e.g., inspection by using the HD03 hemodialysis monitor) to establish a database which may be beneficial to medical professionals during the process of AV fistula assessment.

Since resources of the VGG-19 model are available on the Internet, users are able to revise the source codes of the VGG-19 model and train the VGG-19 model based on their needs. Consequently, the costs of development and maintenance maybe reduced, and results of the blood flow assessment may be relatively credible.

In the description above, for the purposes of explanation, numerous specific details have been set forth in order to provide a thorough understanding of the embodiment. It will be apparent, however, to one skilled in the art, that one or more other embodiments maybe practiced without some of these specific details. It should also be appreciated that reference throughout this specification to “one embodiment,” “an embodiment,” an embodiment with an indication of an ordinal number and so forth means that a particular feature, structure, or characteristic may be included in the practice of the disclosure. It should be further appreciated that in the description, various features are sometimes grouped together in a single embodiment, figure, or description thereof for the purpose of streamlining the disclosure and aiding in the understanding of various inventive aspects, and that one or more features or specific details from one embodiment may be practiced together with one or more features or specific details from another embodiment, where appropriate, in the practice of the disclosure.

While the disclosure has been described in connection with what is considered the exemplary embodiment, it is understood that this disclosure is not limited to the disclosed embodiment but is intended to cover various arrangements included within the spirit and scope of the broadest interpretation so as to encompass all such modifications and equivalent arrangements.

What is claimed is:

1. A system for blood flow assessment in an arteriovenous (AV) fistula, said system comprising:
a radio device including
a transmitting antenna,
a receiving antenna,
a transmitting module that is configured to cooperate with said transmitting antenna to emit a carrier radio wave toward the AV fistula, and
a receiving module that is configured to receive, via said receiving antenna, a return wave signal which is formed through reflection of the carrier radio wave by the AV fistula, and to output a transmission signal which is generated based on the return wave signal; and
a processing device including
a communication module that is in signal connection with said receiving module, and that is configured to receive the transmission signal, and to recover a digital signal from the transmission signal,
a digital filtering module that is in signal connection with said communication module, and that is configured to receive the digital signal, to perform a digital filtering process on the digital signal to result in a filtered signal, and to output the filtered signal, and
a recognition module that is in signal connection with said digital filtering module, and that is configured to receive the filtered signal, to generate a plurality of graphic files based on a waveform of the filtered signal, to perform image recognition on the graphic files, and to output a result of the image recognition as a result of the blood flow assessment of the AV fistula.

2. The system as claimed in claim 1, wherein:
said processing device further includes a differentiation module that is in signal connection with said communication module, and that is configured to receive the digital signal, to perform differentiation on the digital signal, and to output the digital signal thus differentiated; and
said digital filtering module is in signal connection with said differentiation module, and is configured to receive the digital signal thus differentiated, and to perform the digital filtering process on the digital signal thus differentiated to result in the filtered signal.

3. The system as claimed in claim 2, wherein said differentiation module is configured to perform differentiation on the digital signal with respect to time.

4. The system as claimed in claim 1, wherein said receiving module includes:
a demodulation and filtering circuit that is electrically connected to said receiving antenna, and that is configured to receive the return wave signal, to perform demodulation and filtering on the return wave signal to result in a demodulated signal, and to output the demodulated signal;
an analog-to-digital converter that is electrically connected to said demodulation and filtering circuit, and that is configured to receive the demodulated signal, to perform an analog-to-digital conversion on the demodulated signal to result in the digital signal, and to output the digital signal; and
a transmission circuit that is electrically connected to said analog-to-digital converter, and that is configured to receive the digital signal, to transform the digital signal into the transmission signal, and to output the transmission signal.

5. The system as claimed in claim 1, wherein said digital filtering module is configured to pass a part of the digital signal with a frequency ranging from 0.2 Hz to 10 Hz as the filtered signal.

6. The system as claimed in claim 1, wherein said recognition module is a server, includes a database that is configured to store in advance a convolutional neural network (CNN) model, and is configured to perform image recognition on the graphic files by using the CNN model.

7. A method for blood flow assessment in an arteriovenous (AV) fistula, to be implemented by a system for blood flow assessment in an AV fistula, said method comprising:
A) emitting a carrier radio wave toward the AV fistula, and receiving a return wave signal which is formed through reflection of the carrier radio wave by the AV fistula;
B) generating a transmission signal based on the return wave signal;
C) recovering a digital signal from the transmission signal, performing a digital filtering process on the digital signal to result in a filtered signal, and generating a plurality of graphic files based on a waveform of the filtered signal; and
D) performing image recognition on the graphic files, and outputting a result of the image recognition as a result of the blood flow assessment of the AV fistula.

8. The method as claimed in claim 7, wherein step C) includes performing differentiation on the digital signal, and performing the digital filtering process on the digital signal thus differentiated to result in the filtered signal.

9. The method as claimed in claim 8, wherein step C) includes performing differentiation on the digital signal with respect to time.

10. The method as claimed in claim 7, wherein step C) further includes:
C1) computing a heartbeat sampling number based on a heart rate of a subject, and determining, for every heartbeat sampling number of discrete data points of the filtered signal, an extremum of the filtered signal from among the discrete data points; and
C2) dividing, based on the extrema thus determined, the filtered signal into a plurality of signal segments, and generating the graphic files based on waveforms of the signal segments, respectively.

11. The method as claimed in claim 7, wherein step D) includes performing image recognition on the graphic files by using a convolutional neural network (CNN) model.

12. The method as claimed in claim 7, wherein step D) includes performing image recognition on the graphic files by using a Visual Geometry Group-19 (VGG-19) model.
